(19) United States
(12) Patent Application Publication
(10) Pub. No.: US 2010/0179418 A1
(43) Pub. Date: Jul. 15, 2010

(54) INSTRUMENT ALIGNING METHOD USING A FREE REFERENCE

(76) Inventors: Matthias MUELLER, Berlin (DE); Florian GRAS, Jena (DE); Georg CHRISTIAN, Munich (DE); Jörg UHDE, Munich (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/483,342

(22) Filed: Jun. 12, 2009

Related U.S. Application Data
(60) Provisional application No. 61/088,373, filed on Aug. 13, 2008.

(30) Foreign Application Priority Data
Jun. 16, 2008 (EP) 08 010 889.7

(51) Int. Cl. A61B 5/05

(52) U.S. Cl. 600/424

(57) ABSTRACT

The invention relates to an instrument aligning method in which a medical instrument to be aligned is aligned to a target point, wherein the spatial position of the instrument to be aligned is determined and tracked by means of a medical tracking system, wherein the target point is indicated by a freely movable pointing instrument, which is likewise determined and tracked by means of the medical tracking system, and the instrument to be aligned is then aligned to the target point with the aid of a medical navigation which is associated with the tracking system.

### RELATED APPLICATION DATA

This application claims the priority of U.S. Provisional Application No. 61/088,373, filed on Aug. 13, 2008, which is hereby incorporated in its entirety by reference.

### FIELD OF THE INVENTION

The invention relates to an instrument aligning method in a medical setting. In particular, it relates to aligning a medical instrument to a target point, wherein the spatial position of the instrument to be aligned is determined and tracked by means of a medical tracking system.

### BACKGROUND OF THE INVENTION

Navigation-assisted aligning methods for instruments are known in principle. DE 196 39 615 A1 already describes a navigation system in which registered patient image data from imaging methods (CT, MRI, PET, etc.) and tracked instruments are used for example to plan penetration trajectories of instruments or to output them together with patient image data.

One disadvantage of such surgical navigation and planning is that in order to reference and update the assignment of patient image data, a reference array (markers, reference star) which is rigidly fastened to the patient and/or part of the patient's body has to be provided. Such “complete” navigation is also very elaborate, especially with regard to acquiring patient data beforehand. Also, even attaching navigation and/or tracking references is often associated with highly invasive procedures, and because the reference has to be attached in the vicinity of the region to be treated, it can also interfere with the treatment. References which are fastened using elastic bands can lead to inaccuracies, and mechanical aids for “aiming” for medical instruments are often complicated to handle and sometimes also obstructive during the treatment.

### SUMMARY OF THE INVENTION

It is the object of the present invention to provide an instrument aligning method which overcomes at least one of the aforementioned disadvantages of the prior art. The intention is in particular to provide a simple, uncomplicated and/or straightforward aligning method which nonetheless exhibits a high level of accuracy.

This object is solved in accordance with the invention by an instrument aligning method in which a medical instrument to be aligned is aligned to a target point, wherein the spatial position of the instrument to be aligned is determined and tracked by means of a medical tracking system, wherein the target point is indicated by a freely movable pointing instrument, which is likewise determined and tracked by means of the medical tracking system, and the instrument to be aligned is then aligned to the target point with the aid of a medical navigation which is associated with the tracking system. The sub-claims define preferred embodiments of the invention.

In the instrument aligning method of the present invention, the target point is indicated by a freely movable pointing instrument, which is likewise determined (identified and positionally determined) and tracked by means of the medical tracking system, and the instrument to be aligned is then aligned to the target point with the aid of a medical navigation which is associated with the tracking system. In other words, the invention refrains from complicated and elaborate “complete” navigation and utilizes the recognition that in many cases, it is simply only necessary to know a target point, wherein the instrument to be operated (instrument to be aligned, tool) merely has to be aligned to this target point in order to be able to obtain a satisfactory result. The invention thus simply replaces complicated navigation and planning steps with indicating a target point. Such a target point can however be indicated simply and very accurately (to the level of accuracy of the tracking system) using a pointing instrument, such that an instrument can be aligned in a straightforward but accurate way in accordance with the invention.

The method in accordance with the invention and all its embodiments described here can also be regarded as a medical planning method which allows the person carrying out the treatment to verify or estimate the success of a step before it is actually performed, for example the correct alignment of an instrument or tool and therefore also the correct alignment of the treatment result produced using it. The present invention does not directly serve for treating patients but rather for preparing and ensuring the success of a treatment by visually assisting the person carrying out the treatment. The invention can also be interpreted as a system invention or device invention, wherein the devices used in the method and combinations of them are encompassed and assembled in accordance with the invention.

A major advantage and particular aspect of the invention is also that the pointing instrument forms a sort of “free reference” which no longer has to be rigidly or immovably attached to the part of the patient's body, because it is sufficient to be able to locate the tip of the pointing instrument at the target point. Complicated and invasive preparations, or attaching a rigid reference to a part of the patient's body, are therefore no longer necessary.

In one embodiment, the instrument to be aligned can be aligned from a placing point to the target point, wherein the placing point lies on the part of the patient's body and can be freely chosen by the person carrying out the treatment within the bounds of the anatomical conditions and the form of treatment chosen. Many physicians can tell in a simple way or from their experience where an instrument is to be placed; determining its trajectory and exit point, however, is more difficult and it is here that the present invention helps.

As already stated above, the pointing instrument does not have to be fixedly and/or rigidly arranged relative to the part of the patient's body on which the target point and/or placing point lie and/or does not have to be fixedly or rigidly connected to the part of the patient's body. It can preferably be freely guided manually and/or freely handled. In the context of the present disclosure, these terms are not intended to include all instruments which can be manually guided somehow or with great force or at great cost, but rather those instruments which are relatively easily handled freely and those which can be handled freely in accordance with the nature of the instrument. The pointing instrument can specifically be a navigated instrument which comprises a tracking reference and a trackable element and can be moved to a target point. This applies to a pointer comprising a pointer tip, because it is then known where the trackable element, i.e. the tip, lies on the pointer at a distance from the tracking references.

The instrument to be aligned can be tracked by the tracking system directly or however indirectly, for example by tracking a guide for the instrument to be aligned. It can be and/or comprise one or more of the following instruments:
a chisel;
a surgical fraise (a so-called “shaver”);
a screwdriver;
a navigated implant such as for example a nail, a screw or a plate;
a puncture needle;
a trocar;
a drainage.

Within the context of the present disclosure, devices which are medically used are subsumed by the term “instruments”, and the invention can be realized with any instruments for which the alignment is a concern.

In accordance with one embodiment of the present invention, the medical navigation is an image-free navigation, i.e. a navigation for the instruments only, without patient image data or any reference with respect to patient image data. Within the context of this navigation, it is possible to display two-dimensional or three-dimensional trajectory information, angle information and/or distance information in relation to the instruments on the image output of the navigation system. Another option is to display angles, angular deviations or distance values between instrument parts, instrument axes or between geometric properties which are assigned to the instruments. These can be displayed on the image output in numerical form or as diagrams, wherein bar charts or pie charts can specifically be used.

One or a combination of the following reference frames can be chosen as the reference system for the medical navigation and/or for visually displaying it:
the camera reference frame of the tracking system;
a reference system which is defined by one of the instruments;
a reference system which is defined at least partially by relationships between two or more instruments.

In accordance with other aspects, the invention relates to a program which, when it is running on a computer or is loaded onto a computer, causes the computer to perform a method such as has been explained above in variant embodiments, and to a computer program storage medium comprising such a program.

A number of treatments which are medically assisted are based on the object of aiming for a particular point—the target point—and/or aligning an instrument to said point. The present invention then helps in reaching such a target, as is described in the following embodiments which are in turn only example embodiments. The target can be reached via a defined access trajectory which starts at a defined starting point or placing point, or via some other trajectory which passes through the target point. Such a defined access trajectory could for example be a drill hole. The target point itself could be a structure on the surface or below the surface of an articular cartilage or on a bone in a specific region or on a point which is defined by a tool or an implant. Any placing point or starting point then defines a trajectory to the target point, and navigated tools define both points, which are correlated by a subsequent surgical step. The target can then be reached by a defined or selected trajectory, wherein a navigated tool and the pointing instrument, which points towards the target point, are used. A rigidly fastened reference on the part of the patient's body is not necessary. Using an image-free navigation technique according to an embodiment in accordance with the invention, it will thus be possible to use a navigation guide (displaying angle information and depth information) for a navigated tool or implant, without having to attach rigid references, which expedites the navigated use of the tool and placement of an implant.

### BRIEF DESCRIPTION OF THE DRAWINGS

The invention is explained below in more detail on the basis of example embodiments and with the aid of the enclosed drawings. It can comprise any of the features described here, individually and in any expedient combination.

FIG. 1 shows a schematic representation of an instrument aligning method in accordance with the invention; and

FIG. 2 shows schematic representations of a screen output which assists in accordance with the invention.

### DETAILED DESCRIPTION

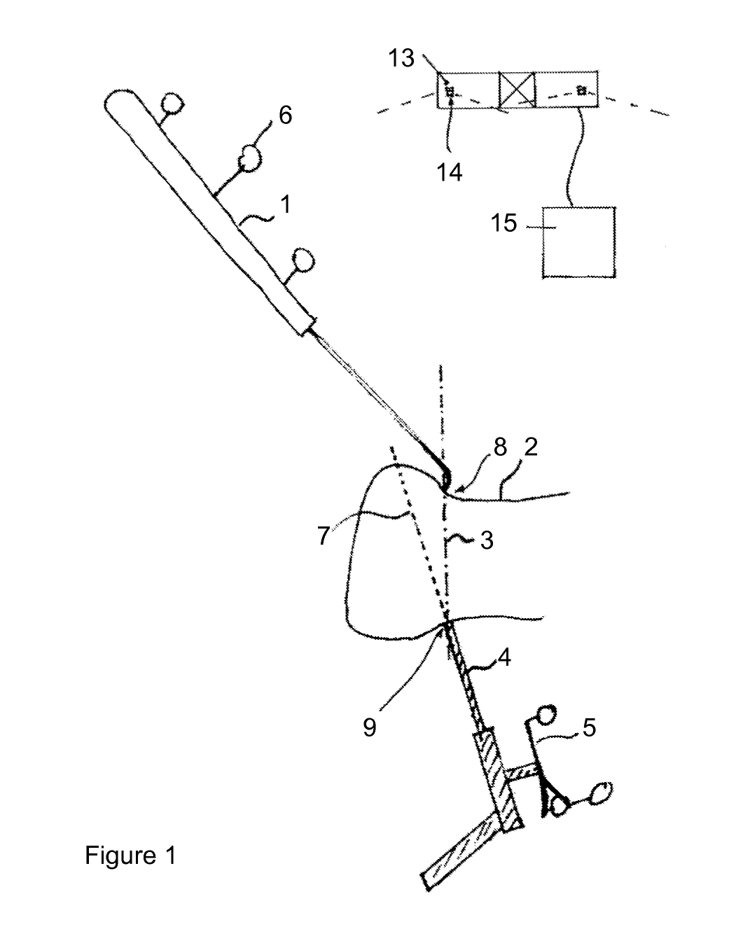

FIG. 1 shows a pointing instrument 1, namely a pointer comprising reference markers on its handle, one of which is indicated by the reference sign 6. The pointer 1 has a tip which is indicated as 8. On the lower side, a drill guide 4 is shown which can likewise be tracked and comprises the tracking reference 5 for this purpose. Both the pointer 1 and the drill guide 4 are tracked by the tracking system 13, which is only schematically shown and comprises two cameras, one of which bears the reference sign 14. The tracking system 13 is integrated into or provided in addition to a medical navigation system, the image output 15 of which is—likewise only schematically—shown. Using the tracking system 13, it is possible to establish the spatial position of the pointer 1 and in particular also of its tip 8, as well as the spatial position of the drill guide 4.

The end of a bone 2 is shown in FIG. 1, merely as an example and representative of any part of a patient's body. It should be stated here that the bone does not necessarily have to lie free in order to perform the present alignment in accordance with the invention; the invention can be perfectly well performed without an invasive incision having been made beforehand on the part of the patient's body.

A drill hole is to be drilled through the bone 2, the axis of which bears the reference sign 3 in FIG. 1. The extended drill guiding sleeve is likewise indicated as an axis and has received the reference sign 7 in the representation in FIG. 1.

In the current alignment, in which the tip of the drill guide 4 has been placed at the placing point 9 on the bone, the axis 7 and therefore the direction of the drill hole would not lead to the target point, i.e. to the point indicated by the pointer tip 8. If the navigation is then used—and only the navigation of the instruments, without any data for the part of the patient's body—it is possible, by moving and rotating the drill guide 4 around the placing point 9, to ensure that the axis 7 matches the desired drill hole axis 3. On the basis of their anatomical knowledge, the operating person knows that no structures lie on the path between the placing point and the target point which would be endangered or permanently damaged by the procedure, or ensures this beforehand. Patient body data is therefore also not required for this navigation. It is merely necessary to know the placing point (point 9, obtained from tracking the drill guide 4) and the target point (point 8, obtained from tracking the pointer tip), and it is possible, by simply displaying the navigation, to plan, verify and show whether the drill sleeve is lying in the correct direction, such that an inserted drill can reach the target point 8.

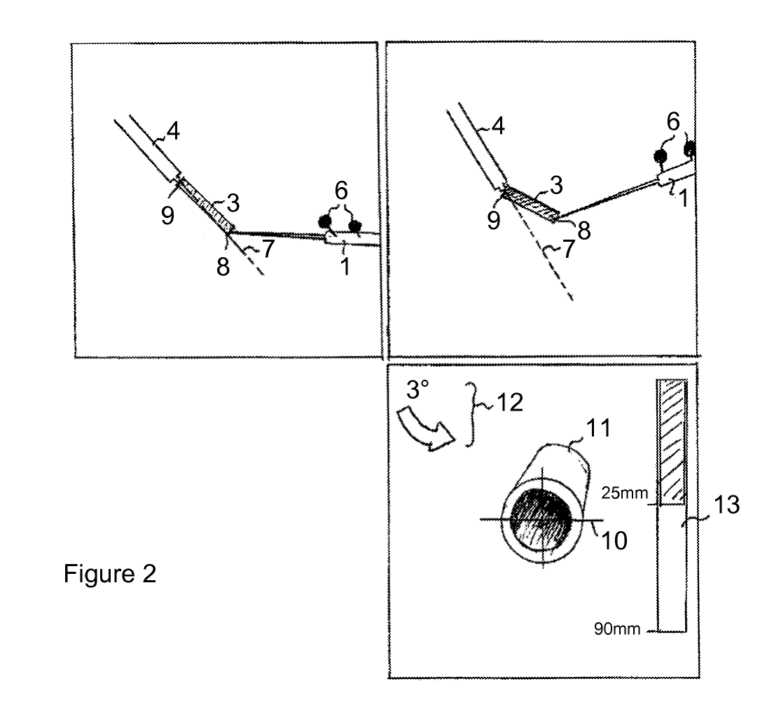

This may be explained even further on the basis of FIG. 2. FIG. 2 shows three representations such as would for example appear on the navigation screen 15 in order to assist the person carrying out the treatment. It can be seen here that there is no patient data displayed, but rather merely images of the navigated pointer 1 and the navigated drill guide 4. As an aid, the desired drill hole 3 can then be superimposed between the tips of the two instruments, and in the upper left-hand representation, it can be seen in one viewing direction that the axis 7 is already almost central in the virtual drill hole 3, such that only a small rotational correction is necessary in this plane. The top-right image in FIG. 2 then shows a view which is offset with respect to this by 90°, and from which it can be seen that in this plane, a relatively large rotation of the drill guide 4 is still necessary in order for it to come to rest centrally in the virtual and desired representation of the drill hole, between the points 8 and 9.

A representation such as can be seen bottom right in FIG. 2 can then for example also be recorded, as an aid to the user for aligning the tool, wherein the centre of a three-dimensional virtual tubular representation of the drill hole 11 is indicated by crosshairs 10, wherein the continuation 11 of the virtual “drill hole tunnel” projecting backwards will disappear increasingly further behind the front circle, the nearer one comes to the desired alignment. Angular deviations can be displayed by numerical data and direction arrows, as shown top left at the reference sign 12, and the representation can also contain a bar chart 13 which for example notifies the distance from the target point still remaining. This provides the user with a very detailed and easily understood visual assistance, and will enable the user to locate and plan the desired trajectory of the drill hole to a high level of accuracy—without using highly invasive rigid reference arrays.

As already stated above, different reference systems (coordinate systems and/or reference frames) can be used for the visual, assisting representation, and if the camera reference frame is used, absolute directions such as “upwards” or “downwards” can for example be indicated and used, which enables a very simple image assistance. Reference systems which are determined as reference frames by the instruments and/or implants or tools and/or their relationships relative to each other are advantageous if one wishes to prevent the desired trajectories from moving on the screen. It would then be possible to always render such trajectories (for example, the alignment of the drill hole or the alignment of the drill guide) centered and parallel on the surface of the screen in one view, and perpendicular to this in another view.

A combination of camera reference frames and instrument relative reference frames could have an advantageous effect when a non-moving trajectory is to be combined with defined directions. Autopilot views could also be used to assist in guiding the tool.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiment(s).

Although the invention has been shown and described with respect to one or more particular preferred embodiments, it is clear that equivalent amendments or modifications will occur to the person skilled in the art when reading and interpreting the text and enclosed drawing(s) of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the element described, i.e. which is functionally equivalent to it, even if it is not structurally equivalent to the disclosed structure which performs the function in the example embodiment(s) illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. An instrument aligning method in which a medical instrument to be aligned is aligned to a target point, wherein the spatial position of the instrument to be aligned is determined and tracked by means of a medical tracking system, wherein the target point is indicated by a freely movable pointing instrument, which is likewise determined and tracked by means of the medical tracking system, and the instrument to be aligned is then aligned to the target point with the aid of a medical navigation which is associated with the tracking system.

2. The instrument aligning method according to claim 1, wherein the instrument to be aligned is aligned from a placing point to the target point.

3. The instrument aligning method according to claim 1, wherein the pointing instrument is not fixedly and/or rigidly arranged relative to the part of the patient's body on which the target point and/or placing point lie and/or is not connected fixedly or rigidly connected to the part of the patient's body.

4. The instrument aligning method according to claim 1, wherein the pointing instrument can be freely guided manually and/or freely handled.

5. The instrument aligning method according to claim 1, wherein the pointing instrument is a navigated instrument which comprises a tracking reference and a trackable element and can be moved to a target point.

6. The instrument aligning method according to claim 5, wherein the pointing instrument is a pointer comprising a pointer tip.

7. The instrument aligning method according to claim 1, wherein the instrument to be aligned is tracked by the tracking system directly.

8. The instrument aligning method according to claim 1, wherein the instrument to be aligned is tracked by the tracking system indirectly.

9. The instrument aligning method according to claim 8, wherein a guide for the instrument to be aligned is tracked.

10. The instrument aligning method according to claim 1, wherein the instrument to be aligned is and/or comprises one of the following instruments:
a medical and/or surgical drill;
a guiding sleeve for a medical and/or surgical drill;
a chisel;
a surgical fraise (a so-called “shaver”);
a screwdriver;
a navigated implant such as for example a nail, a screw or a plate;
a puncture needle;
a trocar;
a drainage.

11. The instrument aligning method according to claim 1, wherein the medical navigation is an image-free navigation, i.e. a navigation for the instruments only, without patient image data or any reference with respect to patient image data.

12. The instrument aligning method according to claim 1, wherein two-dimensional and/or three-dimensional trajectory information, angle information and/or distance information in relation to the instruments is displayed on the image output of the navigation system within the context of the medical navigation.

13. The instrument aligning method according to claim 1, wherein angles, angular deviations or distance values between instrument parts, instrument axes or between geometric properties assigned to the instruments are displayed on the image output of the navigation system in numerical form or as diagrams.

14. The instrument aligning method according to claim 13, wherein the diagrams are bar charts.

15. The instrument aligning method according to claim 1, wherein one or a combination of the following reference frames are chosen as the reference system for the medical navigation and/or for visually displaying it:
the camera reference frame of the tracking system;
a reference system which is defined by one of the instruments;
a reference system which is defined at least partially by relationships between two or more instruments.

16. A program which, when it is running on a computer or is loaded onto a computer, causes the computer to perform a method in accordance with claim 1.

17. A computer program storage medium comprising a program according to claim 16.
